(19) United States
(12) Patent Application Publication
(10) Pub. No.: US 2007/0122361 A1
(43) Pub. Date: May 31, 2007

(54) Tooth colorant and whitener, method of manufacture, and method of use thereof

(76) Inventor: Weitao Jia, Wallingford, CT (US)

Correspondence Address: CANTOR COLBURN, LLP, 55 GRIFFIN ROAD SOUTH, BLOOMFIELD, CT, 06002, US

(21) Appl. No.: 11/289,764

(22) Filed: Nov. 29, 2005

(51) Int. Cl. A61K 8/18; A61K 8/81

(52) U.S. Cl. 424/53; 523/122

(57) ABSTRACT

A polymerizable tooth colorant composition, comprising: a polymerizable resin composition; an additive composition comprising a colorant, a whitener, or both; and a curing system. The composition allows easy coloring and/or whitening of teeth.

### BACKGROUND

This invention relates to tooth colorants, including whitening systems, in particular colorant and whitening glazes, their method of manufacture, and method of use.

Preserving and enhancing the color of teeth and of dental restorations has become popular in recent years. Such treatment is often to reverse or ameliorate discoloration that can arise from a number of sources, for example, treatment with tetracycline medication, fluorine poisoning, trauma and/or death of the tooth, and staining from tobacco use or from food such as coffee or curry.

A common whitening process involves bleaching the teeth using a peroxide-containing or oxygen-generating agent. These are often lengthy and complicated processes, requiring repeated treatment, and are expensive to maintain because peroxide compositions do not prevent subsequent discoloration. Certain patients are also sensitive to the peroxide or other components of the compositions. Another common technique for coloring or whitening a tooth that may be deeply discolored is to remove a thin layer of the tooth surface, then to bond a ceramic or composite veneer to the tooth. Alternatively, a dental restorative composition such as a flowable composite material can be applied directly to the prepared tooth surface to mask the discolored tooth and restore the tooth to a desirable shade. However, these restoration techniques are time-consuming and expensive.

There accordingly remains a need in the art for improved compositions and methods for coloring or whitening teeth. It would further be advantageous if the composition also aids in preventing subsequent tooth discoloration.

### SUMMARY

A polymerizable tooth colorant composition comprises a polymerizable resin composition; a cure system; and a colorant, a whitener, or both, wherein the tooth colorant composition has a viscosity at 25° C. of about 0.1 to about 100 Pa-s.

Another embodiment is a method of manufacturing a polymerizable tooth colorant composition, comprising combining a polymerizable resin composition, a cure system, and a colorant, a whitener, or both.

A method of coloring a tooth comprises preparing the surface of the tooth; applying a polymerizable tooth colorant composition comprising a polymerizable resin composition; a cure system; and a colorant, a whitener, or both to the prepared tooth surface; and curing the applied tooth colorant composition.

### DETAILED DESCRIPTION OF PREFERRED EMBODIMENTS

It has unexpectedly been discovered that an easy to use, relatively inexpensive method to color or whiten teeth can be achieved with a tooth colorant glaze comprising a polymerizable resin composition, a cure system, and a tooth colorant, a whitener, or both. The composition can further optionally have an optical opacifier, an X-ray opacifier, a fluorescer, or a combination comprising at least one of the foregoing. The glaze can be applied without first reducing tooth structure, and is long lasting. The patient undergoing the treatment is thus spared the anxiety, discomfort, and cost of preparatory tooth grinding. The glazes can further be formulated to be stain resistant, thereby decreasing or eliminating the need for re-treatment. As used herein a “tooth colorant composition” refers to a composition that changes the color of a tooth and/or whitens (lightens) the tooth. A “tooth” as used herein includes both natural dentition and dentition that has been restored with a composite or ceramic material.

A wide variety of polymerizable resin compositions can be used to formulate the tooth colorant composition. Such resins contain polymerizable functionalities such as epoxy groups and ethylenically unsaturated groups, for example vinyl groups, acrylate groups, and methacrylate groups. As used herein, the term “(meth)acrylate” encompasses both acrylate and methacrylate groups. The resins are selected so as to provide a coatable composition after incorporation of the other glaze components, that is, a relatively flowable composition that can be applied to a tooth surface with a brush, swab, cannula, or the like. Often a combination of different resins and monomers are used in order to allow ready adjustment of the properties of the curable composition such as viscosity, wettability, shrinkage upon cure, cure speed, and the like, as well as the final properties of the composition, for example hardness, water absorption, stain resistance, and the like. A photocurable composition is preferred, due to the fast cure and ease of use.

(Meth)acrylate resins are preferred, based on their ready availability and ease of polymerization. Known viscous (meth)acrylate resins that can be used in the polymerizable dental resin composition include, for example aliphatic and aromatic polyurethane dimethacrylates (PUDMA), aliphatic and aromatic diurethane dimethacrylates (DUDMA), and the polycarbonate dimethacrylate (PCDMA) disclosed in U.S. Pat. Nos. 5,276,068 and 5,444,104 to Waknine, which is the condensation product of two parts of a hydroxyalkylmethacrylate and 1 part of a bis(chloroformate). Another advantageous resin having lower water sorption characteristics is an ethoxylated bisphenol A dimethacrylate (EBPDMA) as disclosed in U.S. Pat. No. 6,013,694. Another useful (meth)acrylate resin is the condensation product of bisphenol A and glycidyl methacrylate, 2,2′-bis [4-(3-methacryloxy-2-hydroxy propoxy)-phenyl]-propane (Bis-GMA). These viscous resins have a viscosity of greater than about 1000 centipoise (cps) at 60° C.

Relatively low viscosity resins can also be used, that is, resins having a viscosity of about 100 to about 1000 cps at 60° C. A number of aromatic or aliphatic polyurethane(meth)acrylates are commercially available having a viscosity of about 100 to about 1000 centipoise (cps) at 60° C., specifically about 100 to about 500 cps at 60° C.

The above resins can be used with low viscosity diluent monomers having a viscosity of about 0.1 to about 200 cps at 25° C. Suitable diluent monomers include monofunctional or multifunctional (meth)acrylates having a viscosity of about 0.1 to about 100 cps at 25° C. Use of multifunctional (meth)acrylates can increase cure speed of the resin composition. Suitable diluent monomers include those known in the art such as hydroxy alkyl methacrylates, for example 2-hydroxyethyl methacrylate and 2-hydroxypropyl methacrylate; ethylene glycol methacrylates, including ethylene glycol methacrylate, diethylene glycol methacrylate, tri(ethylene glycol)dimethacrylate and tetra(ethylene glycol)dimethacrylate; and diol dimethacrylates such as butanedimethacrylate, dodecanedimethacrylate, or 1,6-hexanedioldimethacrylate (HDDMA). Tri(ethylene glycol)dimethacrylate (TEGDMA) is also useful.

The relative amount of viscous resin, relatively low viscosity resin and diluent monomers is adjusted to provide the desired flowability and final properties, and will depend on the particular resins used, as well at the type and amount of colorant and/or whitener used. Exemplary ratios of relatively low viscosity resin:diluent monomer are 99:1 to 1:99 by weight, more specifically 95:5 to 50:50, still more specifically 85:15 to 70:30 by weight.

The polymerizable tooth colorant composition further comprises a curing system effective for cure of the polymerizable resin composition. Curing systems generally include a polymerization initiator and a polymerization accelerator. Cure systems for epoxy-functional resins can comprise, for example, a ternary photoinitiator system comprising an iodonium salt, a visible light sensitizer, and an electron donor compound.

Preferred curing systems for resins containing ethylenically unsaturated groups include a polymerization photoinitiator and a polymerization accelerator. Either ultraviolet (UV)-activated cure or visible light-activated cure (approximately 230 to 750 nm) is acceptable. Suitable polymerization photoinitiators include visible light activated photoinitiators such as DL-camphorquinone (CQ), and benzil diketones. UV-activated photoinitiator include compounds such as benzil, benzoin, benzoin methyl ether and others. A useful type of commercially available photoinitiators is available under the trade name IRGACURE, from Ciba Specialty Chemicals, and includes for example, phosphine oxides such as bisacylphosphine oxide and trimethylbenzoyldiphenylphosphine oxide. These can be photoinitiated by conventional halogen-type of dental cure lights. The amount of photoinitiator is selected according to the curing rate desired. A minimal catalytically effective amount is generally about 0.01 wt % of the total resin composition, and will lead to a slower cure. Faster rates of cure are achieved with amounts of catalyst in the range from greater than about 0.01 percent to about 5 wt % of the dental composite material.

Alternatively, the polymerizable tooth colorant composition can be formulated as self-curing. Self-cure curing systems generally contain a free radical polymerization initiator such as, for example, a peroxide in an amount of about 0.01 to about 1.0 wt % of the total resin composition. Particularly suitable free radical initiators are lauryl peroxide, tributyl hydroperoxide and, more particularly benzoyl peroxide. Self-curing compositions are generally provided in two parts that are mixed just prior to use, one part containing the free radical initiator and one part containing the polymerization accelerator. Self-curing systems may be combined with light curing systems to provide a dual-cure product. Dual-cure systems (light cure and self cure) can also be used.

Polymerization accelerators suitable for use in any of the above systems include various organic tertiary amines well known in the art. In UV and visible light curing systems, the tertiary amines are generally acrylate derivatives such as dimethylaminoethyl methacrylate and, particularly, diethylaminoethyl methacrylate (DEAEMA) in an amount of about 0.05 to about 0.5 wt % of the resin composition. In the self-curing systems, the tertiary amines are generally aromatic tertiary amines, preferably tertiary aromatic amines such as ethyl 4-(dimethylamino)benzoate (EDMAB), 2-[4-(dimethylamino)phenyl]ethanol, N,N-dimethyl-p-toluidine (DMPT), and bis(hydroxyethyl)-p-toluidine. Such accelerators are generally present in an amount of about 0.5 to about 4.0 wt % of the resin composition.

The curing systems can further comprise an ultraviolet absorber in an amount of about 0.05 to about 5.0 wt % of the resin composition. Such UV absorbers are particularly desirable in the visible light curing systems, in order to avoid discoloration of the resin from incident ultraviolet light. Suitable UV absorbers are the various benzophenones, particularly UV-5411 and Tinuvin P, available from American Cyanamid Company and Ciba Specialty Chemicals Corp., respectively.

The polymerizable tooth colorant composition further comprises a colorant. A “colorant” as used herein is a substance that can impart a color when applied to a tooth. A “color” thus can be any perceivable hue, tint, or shade, for example those described by L*a*b* color space, as specified by CIELAB (CIE, 1978 and 1986). Five common and recognized indices can be computed from the L*, a* and b* parameters, which can be determined using a calorimeter. The Total Color Difference is the magnitude of the resultant vector of three component differences: L (+ΔL=Lighter), a (+Δa=Redder) and b(+Δb=Yellower). The total magnitude of color difference E (ΔE) between two colors can be determined by calculating the square root of ((ΔL)²+(Δa)²+(Δb)²).

Combinations of colorants are generally used, for example combinations of red, yellow, gray, blue, brown, and the like, through which one can achieve a desired tooth shade. Suitable colorants are FDA-approved pigments and dyes, for example copper oxide, chromium oxide, various yellow, red, or black iron oxides, and organic pigments such as phthalocyanine green, ultramarine blue, FD&C Green No. 1 lake, FD&C Blue No. 2 lake, FD&C Blue No. 1, FD&C Green No. 3, FD&C Red No. 30 lake, FD&C Yellow No. 15 lake, FD&C Red No. 3, FD&C Yellow No. 5, FD&C Yellow No. 6, FD&C Blue No. 4, Red #40, FD&C Red No. 30, Food Red No. 17, disodium salt of 6-hydroxy-5-{(2-methoxy-5-methyl-4-sulphophenyl)azo}-2-naphthalenesulfonic acid, Food Yellow No. 13, the sodium salt of a mixture of the mono and disulphonic acids of quinophthalone or 2-(2-quinolyl)indanedione, and combinations comprising at least one of the foregoing.

A whitener as used herein is an additive that results in an increase in the lightness (+ΔL) of a color. A whitener can be used alone or in combination with a dye or pigment. The whitening agent can be an inorganic particulate material such as TiO₂, ZrO₂, BiOCl, Al₂O₃, SiO₂, ZnO, various calcium phosphates compounds, particularly micro- and nanoscaled calcium apatites, carbonate compounds, or other inorganic particulate materials that provide whiteness to the glaze. Useful particulate materials have an average longest dimension of about 10 micrometers or less, preferably is 1 micrometer or less, and most preferably 0.1 micrometer, down to 1 nanometer. Submicron- or nano-scaled particulates are preferred. Without wishing to be bound by any theory, it is believed that the smaller particulates can yield a more homogenous and uniform coating appearance, and/or a smoother and/or glossier cured surface appearance. Suitable whiteners include particulates such as a BiOCl compound available under the trade name Pearl-Glo® UV (Englehard, N.J.), a submicron sized TiO₂ product available under the code P25 (Degussa Corp), nano-scaled ZrO₂, Al₂O₃, and ZnO particles and certain submicron-sized amorphous silicas such as Aerosil® OX-50 (Degussa). These whiteners are particularly useful as they provide both a whitening or milky effect and an opalescent and/or pearlescent appearance. Alternatively, organic whiteners can also be used, for example particulate polyethylene, polypropylene, ethylene/propylene copolymer, polytetrafluoroethylene, or polyhexafluoropropylene. Specific examples of white polymers include polyethylene PE220, polypropylene, and polytetrafluoroethylene (PTFE), as supplied by PreSperse, Inc. (Somerset N.J.).

The tooth colorant composition can further comprise an opacifier and/or a fluorescer. Opacifying agents are particulate materials having a refractive index (RI) that differs from that of the resin composition. Many of the above-mentioned whitening agents can also be considered opacifying agents.

Fluorescers are materials that provide an illuminating florescence effect when the material is exposed to UV light. Exemplary fluorescent agents include 5-thiophenediylbis(5-tert-butyl-1,3-benzoxazole, available under the trade name UVITEX OB from Ciba.

The tooth colorant composition can further comprise a viscosity modifier, which as used herein includes a thixotropic agent. Fumed silica, for example, is known to impart thixotropic properties to flowable dental resin compositions. Also suitable as a viscosity-modifying additive is a nanosized polyhedral oligomeric silsesquioxane (POSS) filler, and/or a nanosized filler derived from a sol-gel process, optionally together with other conventional dental composite filler materials as taught in U.S. Pat. Nos. 6,417,246; 6,653,365; and 6,787,629; U.S. patent application Ser. No. 10/665,391 filed Sep. 19, 2003; U.S. patent application Ser. No. 10/452,269 filed Jun. 2, 2003; and U.S. patent application Ser. No. 10/683,750, filed Oct. 10, 2003, all of which are incorporated herein by reference in their entirety.

Polyhedral oligomeric silsesquioxane fillers are of the generic formula (RSiO_{3/2})ₙ, wherein R is a hydrocarbon and n is 6, 8, 10, 12, or higher. Such POSS materials are commercially available, for example from Hybrid Plastics. In one embodiment, R is a C₁-C₂₄ straight, branched, or cyclic alkyl group, or a C₆-C₂₄ aromatic, alkylaryl, or arylalkyl group, wherein the alkyl or aromatic groups are optionally substituted with C₁-C₆ alkyl, halo, C₁-C₆ alkoxy, C₁-C₆ perhaloalkyl, and the like. Specific exemplary groups include, phenyl, isooctyl, cyclohexyl, cyclopentyl, isobutyl, or other groups. Such silsesquioxanes include, for example, dodecaphenyl-POSS, octaisooctyl-POSS, octacyclohexyl-POSS, octacyclopentyl-POSS, octaisobutyl-POSS and the like. POSS typically have surface areas greater than 400 square meters per gram (m²/gm).

Polyhedral oligomeric silsesquioxanes as used herein further includes monomers of the general formula Rₙ₊ₚTₙ₋ₚ₋₁Dₚ(OL)ₚ, wherein R is as defined above, T is SiO_{3/2}, D is SiO_{2/2}, L is hydrogen or a hydrocarbon comprising a functional group, and p is a multiple of 3. An exemplary compound of this type has the formula R₇T₄D₃(OL)₃. In a specific embodiment, each R is a phenyl group and each L is a hydrogen, providing a compound of the formula (Ph)₇(SiO_{1.5})₄D₃(OH)₃. One such filler material is commercially available from Hybrid Plastics, Inc. under the designation POSS SO 1458. In another specific embodiment, L is an organic group containing a functional group that may or may be reactive with a component of the resin composition, for example a functional group such as a halide, alcohol, amine, isocyanate, acid, acid chloride, silanol, silane, (meth)acrylate, olefin, epoxide, and the like. The organic group is a divalent group of 1 to 36 carbon atoms that serves as a linker between the D groups and the functional group.

Other types of functionalized POSS fillers may also be used, and include those of the general formula Rₙ₋ₘTₙFₘ wherein R is a hydrocarbon; n is 6, 8, 10, 12 or higher; m is 1 to n; T is SiO_{1.5}, and F is an organic group comprising a functional group, wherein the functional group includes, for example, halide, alcohol, amine, isocyanate, acid, acid chloride, silanols, silane, acrylate, methacrylate, olefin, epoxide, and the like. One, two, or more of the functional groups may be reactive with at least one component of the resin composition. In some cases, it is possible to have all of the covalently bound organic groups be reactive groups.

Such compounds, may be prepared, for example, by corner-capping an incompletely condensed POSS containing trisilanol groups with a substituted trichlorosilane. For example, the trisilanol functionality of R₇T₄D₃(OH)₃, can be reacted with Cl₃Si—F to produce the fully condensed POSS monomer R₇T₈F. Through variation of the F group on the silane, a variety of functional groups can be placed at the corner of the POSS framework, including but not limited to halide, alcohol, amine, isocyanate, acid, acid chloride, silanols, silane, acrylate, methacrylate, olefin, and epoxide.

Preferred functional groups F are acrylate (—X—OC(O)CH═CH₂) and methacrylate (—X—OC(O)CH(CH₃)═CH₂) groups, wherein X is a divalent linking group having 1 to about 36 carbons, such as methylene, ethylene, propylene, isopropylene, butylene, isobutylene, phenylene, and the like. X may also be substituted with functional groups such as ether (e.g., —CH₂CH₂OCH₂CH₂—), as long as such functional groups do not interfere with formation or use of the POSS. X is preferably propylene, isobutylene, or —OSi(CH₃)₂CH₂CH₂CH₂—. One, all, or an intermediate number of the covalently bound groups may be acrylate or methacrylate groups. Such functionalized POSS are available from Gelest, Inc. (Tullytown, Pa.) and Hybrid Plastics. A methacryloxypropyl-substituted T₈ POSS (wherein all positions of the polyhedron are methacryloxypropyl-substituted) is available under the trade designation MA0735 from Hybrid Plastics Corp.). Another methacryloxypropyl-substituted T₈ POSS (wherein one position is methacryloxypropyl-substituted and the remaining positions are isobutyl-substituted) is available under the trade designation MA0702 from Hybrid Plastics Corp (Fountain Valley, Calif.).

The linking groups X are also suitable for use with other functional groups. Other POSS fillers include, for example T₆, T₈, T₁₀, or T₁₂ structures functionalized with alkoxysilanes such as diethoxymethylsilylethyl, diethoxymethylsilylpropyl, ethoxydimethylsilylethyl, ethoxydimethylsilylpropyl, triethoxysilylethyl, and the like; with styrene, such as styrenyl (C₆H₅CH═CH—), styryl (—C₆H₄CH═CH₂) and the like; with olefins such as allyl, —OSi(CH₃)₂CH₂CH₂═CH₂, cyclohexenylethyl, —OSi(CH₃)₂CH═CH₂ and the like; with epoxies, such as 4-propyl-1,2-epoxycyclohexyl, (2-(7-oxa-bicyclo[4.1.0]heptan-3-yl)ethylene, 3-propoxy, glycidyl, (—CH₂CH₂CH₂OCH₂CH(O)CH₂), —OSi(CH₃)₂CH₂CH₂CH₂OCH₂CH(O)CH₂, and the like; with chlorosilanes such as chlorosilylethyl, dichlorosilylethyl, trichlorosilylethyl, and the like; with amines such as aminopropyl, aminoethylaminopropyl, and the like; with alcohols and phenols such as —OSi(CH₃)₂CH₂CH₂CH₂OC(CH₂CH₃)₂(CH₂CH₂OH), 4-propylene-trans-1,2-cyclohexanediol, —CH₂CH₂CH₂OCH₂C(CH₂OH)₂(OH), —OSi(CH₃)₂CH₂CH₂CH₂OC(CH₂OH)₂(CH₂CH₃), and the like; with phosphines such as diphenylphosphinoethyl, diphenylphosphinopropyl, and the like; with norbornenyls such as norbornenylethyl; with nitriles such as cyanoethyl, cyanopropyl, —OSi(CH₃)₂CH₂CH₂CH₂CN, and the like; with isocyanates such as isocyanatopropyl, —OSi(CH₃)₂CH₂CH₂CH₂NCO, and the like, with halides such as 3-chloropropyl, chlorobenzyl (—C₆H₄CH₂Cl), chlorobenzylethyl, 4-chlorophenyl, trifluoropropyl (including a T₈ cube with eight trifluoropropyl substitutions) and the like; and with esters, such as ethyl undecanoat-1-yl and methyl propionat-1-yl, and the like. Certain polymers such as poly(dimethyl-comethylhydrido-co-methylpropyl polymers, poly(dimethyl-comethylvinyl-co-methylethylsiloxy, poly(ethylnorbonenyl-co-norbonene) and poly(ethylsilsesquioxan) may also be used to functionalize POSS. Many of these substitutions are commercially available on T₈ POSS from Hybrid Plastics.

In addition to the POSS and the sol-derived filler, one or more of the inorganic fillers currently used in dental restorative materials may also be present. When present, these fillers are considered to be a part of the “additive composition.” Preferred additional fillers include those that are capable of being covalently bonded to the resin matrix itself or to a coupling agent that is covalently bonded to both. Examples of suitable filling materials include but are not limited to, silica, quartz, strontium silicate, strontium borosilicate, lithium silicate, lithium alumina silicate, amorphous silica, ammoniated or deammoniated calcium phosphate, tricalcium phosphate alumina, zirconia, tin oxide, and Titania. Some of the aforementioned inorganic filling materials and methods of preparation thereof are disclosed in U.S. Pat. No. 4,544,359 and U.S. Pat. No. 4,547,531, pertinent portions of which are incorporated herein by reference. Suitable high refractive index filler materials such as high refractive index silica glass fillers and calcium silicate based fillers such as apatites, hydroxyapatites or modified hydroxyapatite compositions may also be used. Alternatively, inert, non-toxic radiopaque materials such as bismuth oxide (Bi₂O₃), barium sulfate, and bismuth subcarbonate may be used. Suitable fillers have a particle size in the range from about 0.1 to about 5.0 microns, and may further comprise unbound, untreated silicate colloids of about 0.001 to about 0.07 microns. These additional fillers may also be silanized. Commercially available silane-treated fumed silica based on Aerosil A200 can be obtained from Degussa Corp under the names of Aerosil R711 and R7200.

The sum of these additives (colorant, and any opacifier, fluorescer, and/or viscosity modifier) is generally about 30 weight percent (wt. %) or less of the total weight of the polymerizable composition, preferably 20 wt. % or less, more preferably 10 wt. % or less, down to 0.1 wt. % of the total weight of the polymerizable composition.

In contrast to conventional flowable dental restorative composites, such as the compositions taught in the U.S. Pat. No. 6,767,955 Jia et al., the inventive tooth colorant compositions are preferably formulated to have a lower overall viscosity. The lower viscosity contributes to ease of application and formation of a very thin film. Accordingly, the resin composition and additives (colorants and the like) are selected to provide the curable composition a viscosity in the range of about 0.01 to about 100 Pascal-seconds (Pa-s), more specifically about 0.1 about 50 Pa-s, even more specifically about 1 to about 300 Pa-s, and most specifically about 0.1 to about 10 Pa-s, each measured at room temperature (25° C.).

In one method of manufacture, the polymerizable tooth colorant composition is formulated by combining each of the components of the tooth colorant glaze to provide a one-part coating formulation. Alternatively, the components of the dental resin composition and curing system are precombined, and the additives (colorant(s), whitener(s), opacifying agent(s), fluorescer(s), and viscosity modifying agents are precombined. The resin composition/curing system can then be combined with the additive composition to provide a one-part coating formulation. In one embodiment a variety of different colors and shades are provided to the practitioner in the form of a kit.

In still another alternative embodiment, the resin composition/curing system is provided to the practitioner as a first part and the additive composition is provided to the practitioner as a second part, for example in the form of powder or liquid/gel concentrate. The practitioner can then mix the components prior to use. A variety of colorant/whitener shades can be provided, allowing the practitioner to adjust the color and whiteness of the tooth colorant composition as desired. In this embodiment it may be useful to vary the components present in each part, for example to include one or more additives in the resin part and one or more resins in the colorant part, in order to provide ease of mixing. The two parts are metered out and then mixed, for example using a spatula.

In an advantageous feature, there is no need to remove significant amounts of enamel or dentin in order to use the present composition, due at least in part to the low viscosity of the tooth colorant compositions. In practice, the practitioner can simply acid etch the tooth surface using a conventional dentin/enamel etching agent, followed by subsequent water rinse and drying. Alternatively, a self-etching agent/primer can be applied to the tooth surface first, to create tooth bonding favorable surface before accepting the tooth colorant composition described above. In still another embodiment, the tooth colorant composition can be applied as a more temporary coating directly applied onto a tooth without any surface treatment. In such case, the bond between the coating and the tooth surface is less permanent, and the coating can be removed with less effort.

In one embodiment, the composition is a flowable gel or slurry, and thus placed using a brush, single dose capsule, cannula, or similar means. For example, compositions containing lower concentrations of additives may be applied to a dental surface with a cannula, and then spread using a brush, sponge, or other instrument. An applicator such as a brush can be dipped into the tooth colorant composition, and the composition can then be painted onto the tooth. In addition to brush application, other non-limiting modes of application can comprise applying a rinse comprising the fluid, a semi-solid tooth-coating fluid from a stick resembling a lipstick, applying a semi-solid form using a crayon-like stick, spraying on the fluid, dabbing on the fluid using a towelette, or transferring the fluid from adhesive strip. The composition is applied as a thin film, for example a film having a thickness of less than or equal to about 1 mm, preferably less than or equal to about 0.5 mm, and most preferably less than or equal to about 0.1 mm, down to about 0.001 mm. The formation of a very thin film on the treated tooth surface also advantageously provides a comfort level to the patient, in that the overall size of the tooth is not significantly increased.

After placement (or just prior to placement), cure can be initiated through the use of a conventional dental visible light source, for example a halogen or LED curing lamp, an ultraviolet light, or by raising the temperature of the tooth colorant composition.

The invention is further illustrated by the following non-limiting examples. Unless otherwise specified, the amounts of each component of the formulations shown in the Tables are in parts by weight.

### EXAMPLE 1

Tooth colorant glaze formulations comprising the following components were prepared, wherein the amounts shown are parts per hundred.

TABLE 1

| Components | A | B | C | D |
|---|---|---|---|---|
| Hexafunctional aromatic | 78.6 | 78.6 | 78.6 | 78.6 |
| urethane acrylate (MW approx. | | | | |
| 800, CN 975, Sartomer) | | | | |
| Trifunctional acrylate resin | 8.3 | 8.3 | 8.3 | 8.3 |
| (SR 9012, Sartomer) | | | | |
| Tetrahydrofurfurylmethacrylate | 8.5 | 8.5 | 8.5 | 8.5 |
| Gamma-methacryloxypropyl | 1.2 | 1.2 | 1.2 | 1.2 |
| trimethoxysilane | | | | |
| UV absorber (UV-5411) | 0.75 | 0.75 | 0.75 | 0.75 |
| UVITEX-OB (Fluorescent agent) | 0.01 | 0.01 | 0.01 | 0.01 |
| CQ | 0.17 | 0.17 | 0.17 | 0.17 |
| EDMAB | 0.47 | 0.47 | 0.47 | 0.47 |
| 2,4,6-Trimethylbenzoyl- | 2.0 | 2.0 | 2.0 | 2.0 |
| diphenylphosphine oxide | | | | |
| (Lucirin ™ TPO, BASF) | | | | |
| POSS (PM 1271, Hybrid Plastics, | 0 | 2 | 0 | 5 |
| Technical Grade of SO1458) | | | | |
| POSS (MA 0735, Hybrid Plastics) | 0 | 0 | 2 | 0 |

Each of the resin compositions of Table 1 were formed into discs by placing the resin into the interior of a mold having a diameter of 15 millimeters (mm) and thickness of about 1 mm, and that was situated between two glass slides. The resin compositions were cured by exposure to UV light. The cured discs were removed from the mold and used to test for staining resistance. Two disks were prepared for each material for each stain resistance test. In addition to compositions A-D of Table 1, an additional control composition (Sample E) was formed into discs. Sample E was a commercially available, (meth)acrylate crown and bridge light curable glaze containing no filler.

Stain resistance tests were performed by submerging each sample disc halfway into two different solutions for durations of one hour and three 3 days, respectively, in an incubator set at 37° C. The first solution was a coffee solution made using one pre-measured/packed single bag per cup, and the second solution was a 2 weight % curry powder in water. A Seradyn ColorWalk™ colorimeter (Photovolt Instrument) was used to measure the colors of the discs. Before each measurement, the samples were rinsed with tap water and dried. Color measurements were made before submerging the samples into the solutions, after one hour of submersion and after three days of submersion. The color scales of L*, a*, b* were recorded and ΔE was calculated based on the color change between the samples before and after testing. The average ΔE numbers for each test group are presented in Table 2 below. A higher ΔE value (increased color change) means lower stain resistance.

TABLE 2

| | Coffee | Coffee | Curry | Curry |
|---|---|---|---|---|
| | (one hr), | (3 days), | (one hr), | (3 days), |
| Sample | ΔE | ΔE | ΔE | ΔE |
| A* | 2.5 | 3.4 | 11.1 | 42.3 |
| B | 1.2 | 2.4 | 4.9 | 38.2 |
| C | 1.8 | 2.6 | 3.3 | 37.7 |
| D | 1.5 | 2.5 | 3.6 | 31.7 |
| E* | 2.2 | 4.4 | 13.2 | 45.5 |

*Controls

The data of Table 2 shows that the compositions comprising POSS filler surprisingly resisted staining upon both short-term and long-term exposure to coffee and curry better than compositions without the filler.

### EXAMPLE 2

The following Table 3 illustrates use of various whitening fillers in the inventive tooth colorant glaze formulations, wherein the amounts shown are in parts per hundred.

TABLE 3

| | Composition | | | | | | |
|---|---|---|---|---|---|---|---|
| | 1 | 2 | 3 | 4 | 5 | 6 | 7 |
| Composition B* | 99 | 96.5 | 97.8 | 97 | 97 | 97 | 100 |
| BiOCl (Pearl-Glo UV, | 1 | | 0.5 | 3 | | | |
| Eaglelhead) | | | | | | | |
| TiO2 (P25, Degussa) | | 3.5 | 1.7 | | | | |
| Al₂O₃ (15 nanometer | | | | | 3 | | |
| alumina, Nanotech) | | | | | | | |
| ZnO (Nanotek, 25 | | | | | | 3 | |
| nanometer, Nanophase) | | | | | | | |

*Experimental Composition B from Table 1

Each composition shown in Table 3 was made into a disk (0.5 mm thick, 15 mm diameter) as described above. The cured disks were then measured for Opacity and whiteness, as reflected by Color scale L, using a Seradyn ColorWalk™ calorimeter. The data is presented in Table 4. The Opacity is the percentage of the transmitted light being blocked by the disks, and therefore reflects the loss of light due to the disk. An Opacity value of zero value means complete light transmission, and an Opacity value of 100% means the disk is completely opaque, blocking 100% of the transmitted light.

TABLE 4

| | Color Scale/Whiteness | Opacity, % transmitted |
|---|---|---|
| Composition | value L | light blocked |
| 1 | 71.6 | 32 |
| 2 | 86.1 | 86 |
| 3 | 85.4 | 64 |
| 4 | 77.8 | 59 |
| 5 | 70.4 | 24 |
| 6 | 78.5 | 60 |
| 7 | 72.5 | 0 |

As can be seen from Table 4, the addition of various whitening fillers results in an increase in opacity compared to Composition 7, which has no whitening filler. However, it was unexpectedly found that for some whitening fillers, the whitening value L does not necessarily increase (Compositions 1 and 5). Appropriate selection of fillers therefore allows the formulation of tooth colorant compositions that can alter the tooth shade with or without whitening the tooth, and that can provide an opalescence to the tooth with or without adding significant whiteness.

### EXAMPLE 3

Measurement of viscosities at room temperature of some of the glaze formulations were performed using a Rheomat, Model PM 180 (Rheometric Scientific, NJ), and the results are shown in Table 5. Two commercially available conventional flowable composite products were measured as references.

TABLE 5

| | Average Viscosities |
|---|---|
| Materials | (Pascal-Seconds) |
| Simile Flow (flowable composite, Pentron) | 550 |
| Sculpture Flow (crown and bridge flowable | 230 |
| composite, Pentron) | |
| Composition B (from Table 1) | 2.4 |
| Composition 2 (from Table 3) | 3.5 |
| Composition 3 (from Table 3) | 2.1 |
| Composition 4 (from Table 3) | 2.6 |

As can be seen from the above results the inventive compositions have much lower viscosities, which is expected to provide ease of application for the practitioner and comfort to the patient.

The singular forms “a,” “an,” and “the” include plural referents unless the context clearly dictates otherwise. “Optional” or “optionally” means that the subsequently described event or circumstance may or may not occur, and that the description includes instances where the event occurs and instances where it does not. Compounds are described using standard nomenclature. For example, any position not substituted by any indicated group is understood to have its valency filled by a bond as indicated, or a hydrogen atom. The endpoints of all ranges reciting the same property or quantity are independently combinable and inclusive of the recited endpoint. All references are incorporated herein by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended embodiments.

What is claimed is:

1. A polymerizable tooth colorant composition, comprising:
a polymerizable resin composition;
an additive composition comprising a colorant, a whitener, or both; and
a curing system, wherein the tooth colorant composition has a viscosity at 25° C. of about 0.01 to about 100 Pa-s.

2. The tooth colorant composition of claim 1, wherein the polymerizable resin composition comprises resins with ethylenic unsaturation.

3. The tooth colorant composition of claim 1, wherein the resin composition comprises a relatively low viscosity resin having a viscosity of about 100 to about 1000 cps at 60° C., and a diluent monomer composition having a viscosity of about 0.1 to about 200 cps at 25° C.

4. The tooth colorant composition of claim 1, wherein the weight ratio of the relatively low viscosity resin:diluent monomer composition is about 99:1 to about 50:50.

5. The tooth colorant composition of claim 1, wherein the colorant is a pigment.

6. The tooth colorant composition of claim 1, wherein the colorant is a dye.

7. The tooth colorant composition of claim 1, wherein the whitener is an inorganic particulate.

8. The tooth colorant composition of claim 1, wherein the whitener is titania, alumina, or a combination comprising at least one of the foregoing.

9. The tooth colorant composition of claim 1, wherein the additive composition further comprises a visual opacifier, an x-ray opacifier, a fluorescer, a viscosity modifier, or a combination comprising at least one of the foregoing.

10. The tooth colorant composition of claim 1, wherein the opacifier is BiOCl, alumina, or a combination comprising at least one of the foregoing.

11. The tooth colorant composition of claim 1, wherein the viscosity modifier is a POSS filler.

12. The tooth colorant composition of claim 1, wherein the additive composition comprises a colorant; a whitener or a visual opacifier; and a POSS filler.

13. The tooth colorant composition of claim 1, wherein the ratio of resin composition:additive composition is 99.9:0.1 to 70:30 by weight.

14. The tooth colorant composition of claim 1, wherein the ratio of resin composition:additive composition is 99:1 to 90:10 by weight.

15. The tooth colorant composition of claim 1, wherein tooth colorant composition has a viscosity at room temperature of about 0.1 to about 50 Pa-s.

16. The tooth colorant composition of claim 1, wherein tooth colorant composition has a viscosity at room temperature of about 1 to about 30 Pa-s.

17. The tooth colorant composition of claim 1, wherein tooth colorant composition has a viscosity at room temperature of about 0.1 to about 10 Pa-s.

18. A method of manufacturing a polymerizable tooth colorant composition, comprising combining:
a polymerizable resin composition;
a filler composition comprising a colorant, a whitener, or both; and
a curing system, to form a tooth colorant composition having a viscosity at 25° C. of about 0.01 to about 100 Pa-s.

19. A method of coloring a tooth, comprising
applying to the tooth dental restoration a polymerizable tooth colorant composition having a viscosity at 25° C. of about 0.01 to about 100 Pa-s, and comprising
a polymerizable resin composition;
a filler composition comprising a colorant, a whitener, or both; and
a curing system; and
curing the composition.
